(19) United States
(12) Patent Application Publication
(10) Pub. No.: US 2014/0357594 A1
(43) Pub. Date: Dec. 4, 2014

(54) NEW COMPOUNDS

(71) Applicant: GlaxoSmithkline Intellectual Property (No.2) Limited, Brentford, Middlesex (GB)

(72) Inventors: Thomas Francis Hendrickson, Encinitas, CA (US); Koc-Kan Ho, Holladay, UT (US); Michael David Saunders, Sandy, UT (US); Brian John Stevens, Salt Lake City, UT (US); Krzysztof Swierczek, West Jordan, UT (US); Kevin Bret Wright, Cottonwood Heights, UT (US)

(73) Assignee: GlaxoSmithkline Intellectual Property (No.2) Limited, Brentford, Middlesex (GB)

(21) Appl. No.: 14/353,854

(22) PCT Filed: Oct. 23, 2012
(86) PCT No.: PCT/US12/61439

§ 371 Date: Apr. 24, 2014

Related U.S. Application Data
(60) Provisional application No. 61/550,679, filed on Oct. 24, 2011.

(51) Int. Cl. C07H 19/16

(52) U.S. Cl. CPC C07H 19/16
USPC 514/45; 536/27.6

(57) ABSTRACT

This invention relates to heteroaryl compounds, containing a purinyl moiety, that inhibit DNA methyltransferase (DNMT) activity—including DNMT1, DNMT3a, or DNMT3b—useful in the treatment of cancer and hyperproliferative diseases.

### FIELD OF THE INVENTION

The invention relates generally to heteroaryl compounds, containing a purinyl moiety, that inhibit DNA methyltransferase (DNMT) activity, and to compositions and methods related thereto. In particular, the invention relates to heteroaryl compounds, containing a purinyl moiety, that inhibit DNA methyltransferase (DNMT) activity—including DNMT1, DNMT3a, or DNMT3b—useful in the treatment of cancer and hyperproliferative diseases.

### BACKGROUND OF THE INVENTION

Cancer (and other hyperproliferative diseases) is characterized by uncontrolled cell proliferation. This loss of the normal control of cell proliferation often appears as the result of genetic damage to cell pathways that control progress through the cell cycle. Such damage includes abnormal DNA methylation in malignant cells, in particular methylation of tumor suppressor genes. (Robertson, K. D., et al., Oncogene, 2001, 20, 3139-3155; Jones P. A., et al., Nat. Rev. Genet., 2002, 3, 415-428).

DNA methylation is mediated through DNA methyltransferases (DNMTs). DNMT1, 3a and 3b activity in tumors is essential for perpetuating gene silencing in growth-regulating genes. Elevated levels of DNA methyltransferases, in tumors contribute to tumorigenesis by improper de novo methylation and silencing of tumor suppressor genes (Linhart H. G., et al., Genes Dev., 2007, 21, 3110-3122). For example, DNMT3b protein overexpression was reported as an independent prognostic factor for predicting cancer survival in diffused large B-cell lymphoma patients (Amara, K., et al., Cancer Sci., 2010, 101, 1722-1730). In another example, depletion of DNMT3a was shown to suppress cell proliferation and to restore PTEN in hepatocellular carcinoma cells (Zhao Z., et al., J. of Biomed. & Biotech., Volume 2010, Article ID 737535, 10 pages).

It has been proven that DNA hypomethylating agents, such as decitabine, are useful for the treatment of cancers. Inhibition of DNMT function would lead to a DNA hypomethylating stage. Thus, small molecule inhibitors of DNMTs should be useful in the treatment of diseases involving uncontrolled cell proliferation, and in particular of cancers (Sippl, W., et al., Methods and Principles in Medicinal Chemistry Volume 42, Epigenetic Targets in Drug Discovery, Chapter 8, 2009, 163-183).

One approach to design DNMT inhibitors is to mimic the co-factor (L)-S-adenosyl-L-methionine (SAM) or its metabolite (L)-S-adenosyl-L-homocysteine (SAH). Such an approach has been described in various publications (Wehhab A., et al., US2008/0132525; Isakovic L., et al., Bioorg. Med. Chem. Lett., 2009, 19, 2742-2746; Saavedra O. M., Bioorg. Med. Chem. Lett., 2009, 19, 2747-2751). The present invention is focusing on SAM or SAH mimics. These mimics contain a functional group that is suitable to interact with the DNA binding region, in particular the cytosine binding pocket. Similarly, novel functional groups that can serve as adenosine mimics are described. Such small molecules can bind to DNMTs in a covalent or non-covalent manner, and in turn inhibits the enzymes. Such small molecules should be useful for treating diseases involving uncontrolled cell proliferation, in particular for cancer.

US 2008/0132525 and WO 2006/078752 describe inhibitors of DNA methyltransferase. CA 2030875 describes methods and probes for detecting nucleoside transporter and method for producing the probes. U.S. Pat. No. 7,560,467 describes indazolo-tetrahydropyrimidine-carboxamide derivative kinase inhibitors. US 2002/0068734, U.S. Pat. No. 6,472,391 and U.S. Pat. No. 6,750,218 describe nitrogen-containing heterocyclic compounds. U.S. Pat. No. 6,169,088 and U.S. Pat. No. 6,207,667 describe 1,3 diazines with platelet-derived growth factor receptor inhibitory activity.

### SUMMARY OF THE INVENTION

The present invention provides compounds according to formula (I). The present invention provides compounds which are useful in therapy, in particular in the treatment of cancer and hyperproliferative diseases. The compounds of formula may be inhibitors of DNA methyltransferase (DNMT) activity and especially DNMT1, DNMT3a, or DNMT3b and may be useful in the treatment of conditions mediated by DNMT.

According to a first aspect of the invention, there is provided a compound of formula (I):

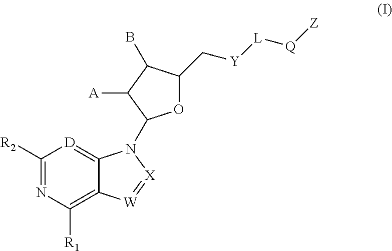

or tautomeric or stereochemically isomeric forms, N-oxides, pharmaceutically acceptable salts or the solvates thereof; wherein:
A and B independently represent hydrogen, halogen or OH;
R₁ represents hydrogen or NR₃R₄;
R₃ and R₄ independently represent hydrogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups;
R₂ represents hydrogen, halogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups;
D represents CH or N;
W represents CH, N, C-alkyl or C-halogen;
X represents CH, N, C-alkyl or C-halogen;
Y and Q independently represent a bond, CR₅R₆, O, S or NR₇;
R₅, R₆ and R₇ independently represent hydrogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups;

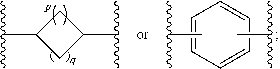

L represents a bond, —(CH₂)ₙ—,
n represents an integer selected from 1 to 5;
p and q independently represents an integer selected from 0 to 3;
Z represents

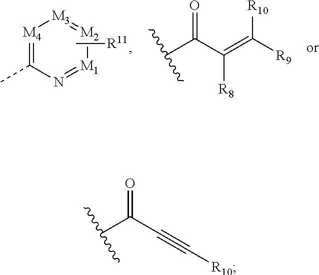

M₁, M₂, M₃ and M₄ independently represent CH or N;
R₈, R₉ and R₁₀ independently represent hydrogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups; and
R₁₁ represents hydrogen, fluorine or chlorine;
Rₐ represents halogen, C₁₋₆ alkyl, C₂₋₆ alkenyl, C₂₋₆ alkynyl, —(CH₂)₀₋₄—C₃₋₈ cycloalkyl, —(CH₂)₀₋₄—C₃₋₈ cycloalkenyl, —(CH₂)₀₋₄-phenyl, —(CH₂)₀₋₄-(heterocyclyl), —(CH₂)₀₋₄-(heteroaryl), —(CR^{x}R^{y})₀₋₄—O—R^{z}, —O—(CR^{x}R^{y})₁₋₄—OR^{z}, haloC₁₋₆ alkyl, haloC₁₋₆ alkoxy, C₁₋₆ alkanol, ═O, ═S, nitro, Si(R^{x})₄, —(CH₂)₀₋₄—CN, —S(O)₀₋₂—R^{x}, —C(═O)R^{x}, —(CR^{x}R^{y})₀₋₄—C(═O)OR^{z}, —(CR^{x}R^{y})₀₋₄—O—C(═O)—R^{z}, —(CR^{x}R^{y})₀₋₄—C(═O)NR^{x}R^{y}, —(CH₂)₀₋₄—NR^{x}C(═O)R^{y}, —(CH₂)₀₋₄—OC(═O)NR^{x}R^{y}, —(CH₂)₀₋₄—NR^{x}C(═O)OR^{y}, —(CH₂)₀₋₄—NR^{x}R^{y}, —NR^{x}—(CH₂)₀₋₄—R^{z}, —(CH₂)₀₋₄—O—C(═O)—C₁₋₄alkyl-NR^{x}R^{y}, —(CH₂)₀₋₄—NR^{x}—(CH₂)₁₋₄—O—C(═O)—R^{z}, —(CH₂)₀₋₄—NR″—(CH₂)₀₋₄—SO₂—R^{y}, —(CH₂)₀₋₄—NH—SO₂—NR^{x}R^{y}, —(CH₂)₀₋₄—SO₂NR^{x}R^{y} and —P(═O)(R^{x})₂ groups; R^{x}, R^{y} and R^{z} independently represent hydrogen, C₁₋₆ alkyl, C₂₋₆ alkenyl, C₂₋₆ alkynyl, —(CH₂)₀₋₄—C₃₋₈ cycloalkyl, —(CH₂)₀₋₄—C₃₋₈ cycloalkenyl, —(CH₂)₀₋₄-(heterocyclyl), —(CH₂)₀₋₄-(heteroaryl), C₁₋₆ alkanol optionally substituted with one or more halo, —C(═O)OC₁₋₆ alkyl, hydroxy, C₁₋₆ alkoxy, haloC₁₋₆ alkyl, —(CH₂)₁₋₄—O—C₁₋₆alkyl, —C(═O)—(CH₂)₁₋₄—C₁₋₆ alkoxy, —C(═O)—C₁₋₆alkyl, —(CH₂)₀₋₄—CN, C₁₋₆ alkyl-N(H)₂₋ᵣ(C₁₋₆alkyl)ᵣ, —N(H)₂₋ᵣ(C₁₋₆alkyl)ᵣ, —C(═O)—N(H)₂₋ᵣ(C₁₋₆alkyl)ᵣ, —(CH₂)₀₋₄—NH—SO₂—N(H)₂₋ᵣ(C₁₋₆alkyl)ᵣ, —(CH₂)₀₋₄—N(C₁₋₄alkyl)-SO₂—N(H)₂₋ᵣ(C₁₋₆alkyl)ᵣ and —(CH₂)₀₋₄—O—C(═O)—C₁₋₄alkyl-N(H)₂₋ᵣ(C₁₋₆alkyl)ᵣ, and when attached to nitrogen or carbon or phosphorus or silicon atom R^{x} and R^{y} may join to form a 3-7 membered ring optionally containing a one or two heteroatoms selected from O, N, S and oxidised forms of N or S; and
r represents an integer selected from 0 to 2.

In a further aspect of the invention there is provided a compound of formula (I) for use in the propylaxis or treatment of a disease or condition as described herein, pharmaceutical compositions comprising a compound of formula (I) and processes for the synthesis of compound of formula (I).

### DEFINITIONS

Unless the context indicates otherwise, references to formula (I) in all sections of this document (including the uses, methods and other aspects of the invention) include references to all other sub-formula, sub-groups, preferences, embodiments and examples as defined herein.

Unless otherwise stated the following terms used in the specification and claims have the meanings discussed below:

“Alkyl” refers to a saturated straight or branched hydrocarbon radical. Examples include methyl, ethyl, propyl, 2-propyl, n-butyl, iso-butyl, tert-butyl, pentyl, hexyl, and the like, preferably methyl, ethyl, propyl or 2-propyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Cyclic alkyls are referred to herein as a “cycloalkyl.” “C₀₋₄alkyl” refers to an alkyl with 0, 1, 2, 3, or 4 carbon atoms. C₀₋₄alkyl with 0 carbon atoms is a hydrogen atom when terminal and is a direct bond when linking.

“Cycloalkyl” refers to a saturated cyclic hydrocarbon radical. Examples include cyclopropyl, cyclobutyl, cyclopentyl and cyclohexyl.

“Halogen” means fluoro, chloro, bromo or iodo, preferably fluoro and chloro.

“Aryl” refers to an all-carbon monocyclic or fused-ring polycyclic (i.e. rings which share adjacent pairs of carbon atoms) groups of 6 to 12 carbon atoms having a completely conjugated pi-electron system. Examples, without limitation, of aryl groups are phenyl, naphthyl and anthracenyl.

“Heteroaryl” refers to a monocyclic or fused ring (i.e. rings which share an adjacent pair of atoms) of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, and carbazole. The heteroaryl group may be substituted or unsubstituted.

“Heterocyclyl” refers to a saturated, unsaturated or aromatic cyclic ring system having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The term “heterocyclyl” includes heteroaryl unless otherwise specified (for example, “saturated heterocyclyl”).

A combination of substituents is permissible only if such as combination results in a stable or chemically feasible compound (i.e. one that is not substantially altered when kept at 40° C. or less for at least a week).

The various functional groups and substituents making up the compounds of the invention are typically chosen such that the molecular weight of the compound of the invention does not exceed 1000. More usually, the molecular weight of the compound will be less than 750, for example less than 700, or less than 650, or less than 600, or less than 550. More preferably, the molecular weight is less than 525 and, for example, is 500 or less.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with the alkyl group.

A “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

“Pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

The term “DNMT mediated condition” or “disease”, as used herein, means any disease or other deleterious condition in which DNMT is known to play a role. The term “DNMT mediated condition” or “disease” also means those diseases or conditions that are alleviated by treatment with a DNMT inhibitor. Such conditions include, without limitation, cancer and other hyperproliferative disorders. In certain embodiments, the cancer is a cancer of colon, breast, stomach, prostate, pancreas, or ovarian tissue.

The term “DNMT activity-mediated condition” or “disease”, as used herein, means any disease or other deleterious condition in which DNMT activity is known to play a role. The term “DNMT activity-mediated condition” also means those diseases or conditions that are alleviated by treatment with a DNMT inhibitor.

The term “treatment” as used herein in the context of treating a condition i.e. state, disorder or disease, pertains generally to treatment and therapy, whether for a human or an animal (e.g. in veterinary applications), in which some desired therapeutic effect is achieved, for example, the inhibition of the progress of the condition, and includes a reduction in the rate of progress, a halt in the rate of progress, amelioration of the condition, diminishment or alleviation of at least one symptom associated or caused by the condition being treated and cure of the condition. For example, treatment can be diminishment of one or several symptoms of a disorder or complete eradication of a disorder.

The term “prophylaxis” (i.e. use of a compound as prophylactic measure) as used herein in the context of treating a condition i.e. state, disorder or disease, pertains generally to the prophylaxis or prevention, whether for a human or an animal (e.g. in veterinary applications), in which some desired preventative effect is achieved, for example, in preventing occurrence of a disease or guarding from a disease. Prophylaxis includes complete and total blocking of all symptoms of a disorder for an indefinite period of time, the mere slowing of the onset of one or several symptoms of the disease, or making the disease less likely to occur.

As used herein, “administer” or “administration” refers to the delivery of an inventive compound or of a pharmaceutically acceptable salt thereof or of a pharmaceutical composition containing an inventive compound or a pharmaceutically acceptable salt thereof of this invention to an organism for the purpose of prevention or treatment of a DNMT-related disorder.

### DETAILED DESCRIPTION OF THE INVENTION

In one embodiment, A and B independently represent hydrogen or OH. In a further embodiment, A and B both represent OH.

In one embodiment, R₁ represents NR₃R₄. In a further embodiment, R₁ represents NH₂.

In one embodiment, R₃ and R₄ independently represent hydrogen or alkyl. In a further embodiment, R₃ and R₄ both represent hydrogen.

In one embodiment, R₂ represents hydrogen, halogen or alkyl. In a further embodiment,

R₂ represents hydrogen.

In one embodiment, D represents N.

In one embodiment, W represents N.

In one embodiment, X represents CH or C-halogen. In a further embodiment, X represents CH.

In one embodiment, Y represents O, S or NR_{S}. In a further embodiment, Y represents S.

In one embodiment, Q represents O, S or NR_{S}. In a further embodiment, Q represents NR_{S}. In a yet further embodiment, Q represents NH.

In one embodiment, R₇ represents hydrogen or alkyl. In a further embodiment, R₇ represents hydrogen.

In one embodiment, L represents —(CH₂)ₙ—. In a further embodiment, L represents —(CH₂)—, —(CH₂)₂— or —(CH₂)₃—. In a yet further embodiment, L represents —(CH₂)₂—.

In one embodiment, n represents an integer selected from 1 to 3. In a further embodiment, n represents 2.

In one embodiment, Z represents

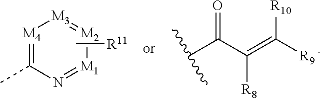

In a further embodiment, Z represents

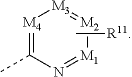

In one embodiment, M₁, M₂ and M₃ represent CH and M₄ represents N. In an alternative embodiment, M₁, M₃ and M₄ represent CH and M₂ represents N.

In a yet further embodiment, Z represents

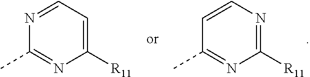

In one embodiment, R₁₁ represents hydrogen or chlorine. In a further embodiment, R₁₁ represents chlorine.

In one embodiment, Z represents

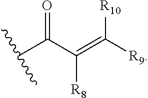

In one embodiment, R₈, R₉ and R₁₀ independently represent hydrogen or alkyl. In a further embodiment, R₈, R₉ and R₁₀ each represent hydrogen.

According to a further aspect of the invention, there is provided a compound of formula (IA) or a pharmaceutically acceptable salt thereof:

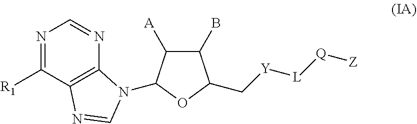

wherein R₁, A, B, Y, L, Q and Z are as defined herein.

According to a further aspect of the invention, there is provided a compound of formula (IB) or a pharmaceutically acceptable salt thereof:

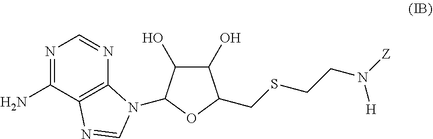

wherein Z is as defined herein.

In one embodiment, the invention provides a compound of formula (I) which is selected from the following compounds or is one of the following compounds:
(2S,3S,4R,5R)-2-(6-amino-9H-purin-9-yl)-5-(((2-((4-chloropyrimidin-2-yl)amino)ethyl)thio)methyl)tetrahydrofuran-3,4-diol (Example 1);
(2S,3S,4R,5R)-2-(6-amino-9H-purin-9-yl)-5-(((2-((2-chloropyrimidin-4-yl)amino)ethyl)thio)methyl)tetrahydrofuran-3,4-diol (Example 2);
(2S,3S,4R,5R)-2-(6-amino-9H-purin-9-yl)-5-(((2-(pyrimidin-4-ylamino)ethyl)thio)methyl)tetrahydrofuran-3,4-diol (Example 3);
(2S,3S,4R,5R)-2-(6-amino-9H-purin-9-yl)-5-(((2-((6-chloropyrimidin-4-yl)amino)ethyl)thio)methyl)tetrahydrofuran-3,4-diol (Example 4);
N-(2-((((2R,3R,4S,5S)-5-(6-amino-9H-purin-9-yl)-3,4-dihydroxytetrahydrofuran-2-yl)methyl)thio)ethyl)acrylamide (Example 5);
   or tautomeric or stereochemically isomeric forms, N-oxides, pharmaceutically acceptable salts or the solvates thereof.

For the avoidance of doubt, it is to be understood that each general and specific preference, embodiment and example for one substituent may be combined with each general and specific preference, embodiment and example for one or more, preferably, all other substituents as defined herein and that all such embodiments are embraced by this application.

### Salts, Solvates, Tautomers, Isomers, N-Oxides, Esters, Prodrugs and Isotopes

A reference to a compound of the formula (I) and sub-groups thereof also includes ionic forms, salts, solvates, isomers (including geometric and stereochemical isomers), tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof, even more preferably the salts or tautomers or solvates thereof.

### Salts

Many compounds of the formula (I) can exist in the form of salts, for example acid addition salts or, in certain cases salts of organic and inorganic bases such as carboxylate, sulfonate and phosphate salts. All such salts are within the scope of this invention, and references to compounds of the formula (I) include the salt forms of the compounds.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, methanesulfonic (mesylate), ethanesulfonic, naphthalenesulfonic, valeric, acetic, propanoic, butanoic, malonic, glucuronic and lactobionic acids. One particular salt is the hydrochloride salt.

If the compound is anionic, or has a functional group which may be anionic (e.g., —COOH may be —COO⁻), then a salt may be formed with an organic or inorganic bases, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the formula (I) contain an amine function, these may form quaternary ammonium salts, for example by reaction with an alkylating agent according to methods well known to the skilled person. Such quaternary ammonium compounds are within the scope of formula (I).

The compounds of the invention may exist as mono- or di-salts depending upon the pKa of the acid from which the salt is formed.

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

In one embodiment of the invention, there is provided a pharmaceutical composition comprising a solution (e.g. an aqueous solution) containing a compound of the formula (I) and sub-groups and examples thereof as described herein in the form of a salt in a concentration of greater than 10 mg/ml, typically greater than 15 mg/ml and preferably greater than 20 mg/ml.

### N-Oxides

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

### Geometric Isomers and Tautomers

Compounds of the formula (I) may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, certain heteroaryl rings can exist in the two tautomeric forms such as A and B shown below. For simplicity, a formula may illustrate one form but the formula is to be taken as embracing both tautomeric forms.

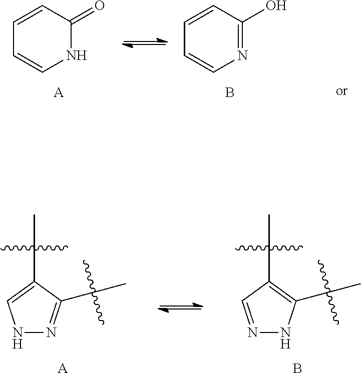

Other examples of tautomeric forms include, for example, keto-, enol-, and enolate-forms, as in, for example, the following tautomeric pairs: keto/enol (illustrated below), imine/enamine, amide/imino alcohol, amidine/enediamines, nitroso/oxime, thioketone/enethiol, and nitro/aci-nitro.

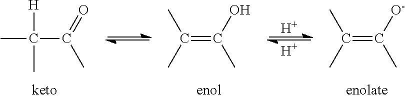

### Stereoisomers

Unless otherwise mentioned or indicated, the chemical designation of compounds denotes the mixture of all possible stereochemically isomeric forms.

Stereocentres are illustrated in the usual fashion, using ‘hashed’ or ‘wedged’ lines. e.g.

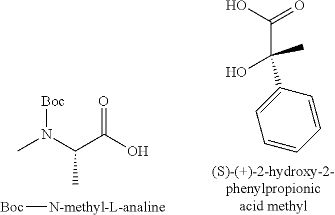

Where a compound is described as a mixture of two diastereoisomers/epimers, the configuration of the stereocentre is not specified and is represented by straight lines.

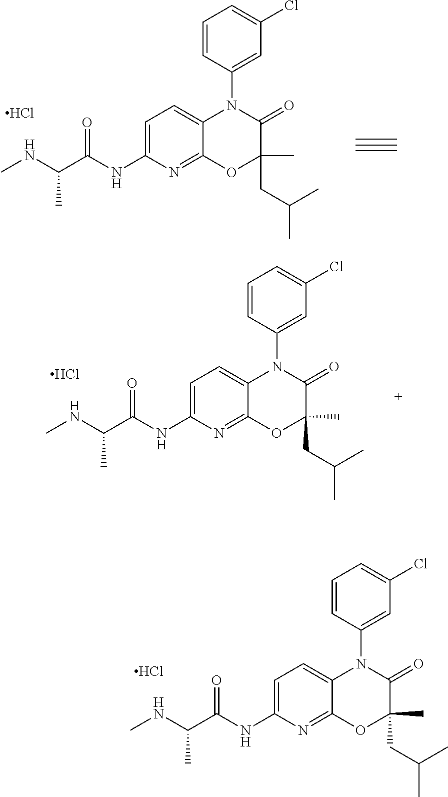

Where compounds of the formula (I) contain one or more chiral centres, and can exist in the form of two or more optical isomers, references to compounds of the formula (I) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulfonic, separating the diastereoisomers by preferential crystallisation, and then dissociating the salts to give the individual enantiomer of the free base.

Additionally enantiomeric separation can be achieved by covalently linking a enantiomerically pure chiral auxiliary onto the compound and then performing diastereisomer separation using conventional methods such as chromatography. This is then followed by cleavage of the aforementioned covalent linkage to generate the appropriate enantiomerically pure product.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). In one general embodiment, 99% or more (e.g. substantially all) of the total amount of the compound of the formula (I) may be present as a single optical isomer (e.g. enantiomer or diastereoisomer).

Compounds encompassing double bonds can have an E (entgegen) or Z (zusammen) stereochemistry at said double bond. Substituents on bivalent cyclic or (partially) saturated radicals may have either the cis- or trans-configuration. The terms cis and trans when used herein are in accordance with Chemical Abstracts nomenclature (J. Org. Chem. 1970, 35 (9), 2849-2867), and refer to the position of the substituents on a ring moiety.

Of special interest are those compounds of formula (I) which are stereochemically pure. When a compound of formula (I) is for instance specified as R, this means that the compound is substantially free of the S isomer. If a compound of formula (I) is for instance specified as E, this means that the compound is substantially free of the Z isomer. The terms cis, trans, R, S, E and Z are well known to a person skilled in the art.

### Isotopic Variations

The present invention includes all pharmaceutically acceptable isotopically-labeled compounds of the invention, i.e. compounds of formula (I), wherein one or more atoms are replaced by atoms having the same atomic number, but an atomic mass or mass number different from the atomic mass or mass number usually found in nature.

Examples of isotopes suitable for inclusion in the compounds of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, and sulfur, such as ³⁶S.

Certain isotopically-labelled compounds of formula (I), for example, those incorporating a radioactive isotope, are useful in drug and/or substrate tissue distribution studies. The compounds of formula (I) can also have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

### Esters

Esters such as carboxylic acid esters, acyloxy esters and phosphate esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₁₂ heterocyclyl group, or a C₅₋₁₂ aryl group, preferably a C₁₋₆ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₆ alkyl group, a C₃₋₁₂ heterocyclyl group, or a C₅₋₁₂ aryl group, preferably a C₁₋₆ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph. Examples of phosphate esters are those derived from phosphoric acid.

In one embodiment of the invention, formula (I) includes within its scope esters of compounds of the formula (I) bearing a carboxylic acid group or a hydroxyl group. In another embodiment of the invention, formula (I) does not include within its scope esters of compounds of the formula (I) bearing a carboxylic acid group or a hydroxyl group.

### Solvates and Crystalline Forms

Also encompassed by formula (I) are any polymorphic forms of the compounds, and solvates such as hydrates, alcoholates and the like.

The compounds of the invention may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include compounds of the invention in combination with water, isopropanol, ethanol, methanol, DMSO, ethyl acetate, acetic acid or ethanolamine and the like. The compounds of the invention may exert their biological effects whilst they are in solution.

Solvates are well known in pharmaceutical chemistry. They can be important to the processes for the preparation of a substance (e.g. in relation to their purification, the storage of the substance (e.g. its stability) and the ease of handling of the substance and are often formed as part of the isolation or purification stages of a chemical synthesis. A person skilled in the art can determine by means of standard and long used techniques whether a hydrate or other solvate has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid State NMR(SS-NMR, also known as Magic Angle Spinning NMR or MAS-NMR). Such techniques are as much a part of the standard analytical toolkit of the skilled chemist as NMR, IR, HPLC and MS.

Alternatively the skilled person can deliberately form a solvate using crystallisation conditions that include an amount of the solvent required for the particular solvate. Thereafter the standard methods described above, can be used to establish whether solvates had formed.

Furthermore, the compounds of the present invention may have one or more polymorph or amorphous crystalline forms and as such are intended to be included in the scope of the invention.

### Complexes

Formula (I) also includes within its scope complexes (e.g. inclusion complexes or clathrates with compounds such as cyclodextrins, or complexes with metals) of the compounds. Inclusion complexes, clathrates and metal complexes can be formed by means of methods well known the skilled person.

### Prodrugs

Also encompassed by formula (I) are any pro-drugs of the compounds of the formula (I). By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl (e.g., -Me, -Et, -nPr, -iPr, -nBu, -sBu, -iBu, -tBu); C₁₋₇aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl).

Also, some prodrugs are activated enzymatically to yield the active compound, or a compound which, upon further chemical reaction, yields the active compound (for example, as in antigen-directed enzyme pro-drug therapy (ADEPT), gene-directed enzyme pro-drug therapy (GDEPT), and ligand-directed enzyme pro-drug therapy (LIDEPT), etc.). For example, the prodrug may be a sugar derivative or other glycoside conjugate, or may be an amino acid ester derivative. In one embodiment formula (I) does not include pro-drugs of the compounds of the formula (I) within its scope.

### Methods for the Preparation of Compounds of Formula (I)

In this section, as in all other sections of this application unless the context indicates otherwise, references to formula (I) also include all other sub-groups and examples thereof as defined herein.

Compounds of the formula (I) can be prepared in accordance with synthetic methods well known to the skilled person.

According to a further aspect of the invention there is provided a process for preparing a compound of formula (I) as hereinbefore defined which comprises:

(a) preparing a compound of formula (I) wherein Q represents NH by reacting a compound of formula (II):

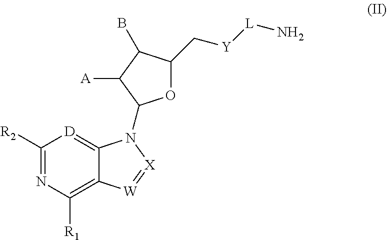

wherein R₁, R₂, D, X, W, A, B, Y and L are as defined hereinbefore for compounds of formula (I), with a compound of formula Z-L₁ wherein Z is as defined hereinbefore for compounds of formula (I) and L₁ represents a suitable leaving group such as chlorine;
(b) deprotection of a protected derivative of a compound of formula (I);
(c) interconversion of a compound of formula (I) or protected derivative thereof to a further compound of formula (I) or protected derivative thereof; and
(d) optional formation of a pharmaceutically acceptable salt of a compound of formula (I).

Process (a) typically comprises reacting the compounds of (II) and Z-L₁ in the presence of a suitable base, such as triethylamine in a suitable solvent, such as ethanol.

Compounds of formula (II) wherein Y represents S and L represents —(CH₂)₂ may be prepared in accordance with the following scheme:

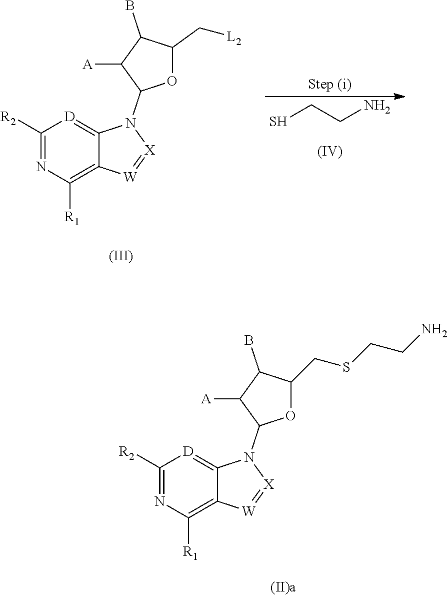

wherein R₁, R₂, D, W, X, A and B are as defined hereinbefore for compounds of formula (I) and L₂ represents a suitable leaving group such as chlorine.

Step (i) typically comprises dissolving the compound of formula (IV) in water and sodium hydroxide followed by bubbling nitrogen through the solution prior to addition of a compound of formula (III). The reaction mixture is then typically heated to reflux prior to cooling to room temperature.

Compounds of formula (III), (IV) and Z-L₁ are either known or may be prepared in accordance with known procedures.

A wide range of well known functional group interconversions are know by a person skilled in the art for converting a precursor compound to a compound of formula I and are described in Advanced Organic Chemistry by Jerry March, 4^{th} Edition, John Wiley & Sons, 1992. For example possible metal catalysed functionalisations such as using organo-tin reagents (the Stille reaction), Grignard reagents and reactions with nitrogen nucleophiles are described in ‘Palladium Reagents and Catalysts’ [Jiro Tsuji, Wiley, ISBN 0-470-85032-9] and Handbook of OrganoPalladium Chemistry for Organic Synthesis [Volume 1, Edited by Ei-ichi Negishi, Wiley, ISBN 0-471-31506-0].

In a further embodiment the invention provides a novel intermediate. In one embodiment the invention provides a novel intermediate of formula (II).

### Protecting Groups

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

A hydroxy group may be protected, for example, as an ether (—OR) or an ester (—OC(═O)R), for example, as: a t-butyl ether; a tetrahydropyranyl (THP) ether; a benzyl, benzhydryl (diphenylmethyl), or trityl (triphenylmethyl)ether; a trimethylsilyl or t-butyldimethylsilyl ether; or an acetyl ester (—OC(═O)CH₃).

An aldehyde or ketone group may be protected, for example, as an acetal (R—CH(OR)₂) or ketal (R₂C(OR)₂), respectively, in which the carbonyl group (>C═O) is treated with, for example, a primary alcohol. The aldehyde or ketone group is readily regenerated by hydrolysis using a large excess of water in the presence of acid.

An amine group may be protected, for example, as an amide (—NRCO—R) or a carbamate (—NRCO—OR), for example, as: a methyl amide (—NHCO—CH₃); a benzyl carbamate (—NHCO—OCH₂C₆H₅, —NH-Cbz or NH—Z); as a t-butyl carbamate (—NHCO—OC(CH₃)₃, —NH-Boc); a 2-biphenyl-2-propyl carbamate (—NHCO—OC(CH₃)₂C₆H₄C₆H₅, —NH-Bpoc), as a 9-fluorenylmethyl carbamate (—NH-Fmoc), as a 6-nitroveratryl carbamate (—NH-Nvoc), as a 2-trimethylsilylethyl carbamate (—NH-Teoc), as a 2,2,2-trichloroethyl carbamate (—NH-Troc), as an allyl carbamate (—NH-Alloc), or as a 2(-phenylsulphonyl)ethyl carbamate (—NH-Psec).

Other protecting groups for amines, such as cyclic amines and heterocyclic N—H groups, include toluenesulphonyl (tosyl) and methanesulphonyl (mesyl) groups, benzyl groups such as a para-methoxybenzyl (PMB) group and tetrahydropyranyl (THP) groups.

A carboxylic acid group may be protected as an ester for example, as: an C₁₋₇ alkyl ester (e.g., a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester; para-methoxybenzyl ester. A thiol group may be protected, for example, as a thioether (—SR), for example, as: a benzyl thioether; an acetamidomethyl ether (—S—CH₂NHC(═O)CH₃).

### Isolation and Purification of the Compounds of the Invention

The compounds of the invention can be isolated and purified according to standard techniques well known to the person skilled in the art and examples of such methods include chromatographic techniques such as column chromatography (e.g. flash chromatography) and HPLC. One technique of particular usefulness in purifying the compounds is preparative liquid chromatography using mass spectrometry as a means of detecting the purified compounds emerging from the chromatography column.

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

Methods of recrystallisation of compounds of formula (I) and salt thereof can be carried out by methods well known to the skilled person—see for example (P. Heinrich Stahl (Editor), Camille G. Wermuth (Editor), ISBN: 3-90639-026-8, Handbook of Pharmaceutical Salts: Properties, Selection, and Use, Chapter 8, Publisher Wiley-VCH). Products obtained from an organic reaction are seldom pure when isolated directly from the reaction mixture. If the compound (or a salt thereof) is solid, it may be purified and/or crystallized by recrystallisation from a suitable solvent. A good recrystallisation solvent should dissolve a moderate quantity of the substance to be purified at elevated temperatures but only a small quantity of the substance at lower temperature. It should dissolve impurities readily at low temperatures or not at all. Finally, the solvent should be readily removed from the purified product. This usually means that it has a relatively low boiling point and a person skilled in the art will know recrystallising solvents for a particular substance, or if that information is not available, test several solvents. To get a good yield of purified material, the minimum amount of hot solvent to dissolve all the impure material is used. In practice, 3-5% more solvent than necessary is used so the solution is not saturated. If the impure compound contains an impurity which is insoluble in the solvent it may then be removed by filtration and then allowing the solution to crystallize. In addition, if the impure compound contains traces of coloured material that are not native to the compound, it may be removed by adding a small amount of decolorizing agent e.g. activating charcoal to the hot solution, filtering it and then allowing it to crystallize. Usually crystallization spontaneously occurs upon cooling the solution. If it is not, crystallization may be induced by cooling the solution below room temperature or by adding a single crystal of pure material (a seed crystal). Recrystallisation can also be carried out and/or the yield optimized by the use of an anti-solvent or co-solvent. In this case, the compound is dissolved in a suitable solvent at elevated temperature, filtered and then an additional solvent in which the required compound has low solubility is added to aid crystallization. The crystals are then typically isolated using vacuum filtration, washed and then dried, for example, in an oven or via desiccation.

Other examples of methods for purification include sublimation, which includes a heating step under vacuum for example using a cold finger, and crystallization from melt (Crystallization Technology Handbook 2nd Edition, edited by A. Mersmann, 2001).

### Pharmaceutical Formulations

While it is possible for the active compound to be administered alone, it is preferable to present it as a pharmaceutical composition (e.g. formulation).

Thus, the present invention further provides pharmaceutical compositions, as defined above, and methods of making a pharmaceutical composition comprising (e.g admixing) at least one compound of formula (I) (and sub-groups thereof as defined herein), together with at least one pharmaceutically acceptable excipient and optionally other therapeutic or prophylactic agents as described herein.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, Pa., USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use.

The pharmaceutical formulation can be prepared by lyophilising a compound of formula (I), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract. Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated. Coatings may act either as a protective film (e.g. a polymer, wax or varnish) or as a mechanism for controlling drug release. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively releasing the compound in the stomach or in the ileum, duodenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The compound of formula (I) may be formulated with a carrier and administered in the form of nanoparticles. Nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer. Ther. August 1, (2006) 5, 1909.

The pharmaceutical compositions typically comprise from approximately 1% (w/w) to approximately 95% (w/w) active ingredient and from 99% (w/w) to 5% (w/w) of a pharmaceutically acceptable excipient or combination of excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) polymers (depending on dose). The film coats of the tablet or capsule typically contain 0-10% (w/w) release-controlling (e.g. delaying) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. In certain embodiments, the preferred routes of administration are oral and intravenous. Alternatively, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with tumor-specific antibody. In this way, the liposomes may be targeted to and taken up selectively by the tumor.

Pharmaceutical compositions of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in any conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also. Pharmaceutical compositions which may also be used include hard gelatin capsules. The capsules or pills may be packaged into brown glass or plastic bottles to protect the active compound from light. The containers containing the active compound capsule formulation are preferably stored at controlled room temperature (15-30° C.).

For administration by inhalation, the compounds for use according to the present invention may be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD cosolvent system. VPD is a solution of 3% w/v benzyl alcohol, 8% w/v of the nonpolar surfactant polysorbate 80, and 65% w/v polyethylene glycol 300, made up to volume in absolute ethanol. The VPD cosolvent system (VPD:D5W) consists of VPD diluted 1:1 with a 5% dextrose in water solution. This cosolvent system dissolves hydrophobic compounds well, and itself produces low toxicity upon systemic administration. Naturally, the proportions of such a cosolvent system may be varied considerably without destroying its solubility and toxicity characteristics. Furthermore, the identity of the cosolvent components may be varied: for example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

### Methods of Treatment

The compounds of the invention have utility over a broad range of therapeutic applications, and may be used to treat diseases, such as cancer, that are mediated and/or associated (at least in part) with DNMT activity.

In another aspect, the invention provides methods for treating or preventing a DNMT activity-mediated disease, such as cancer, which method comprises administering to a patient in need of such a treatment a therapeutically effective amount of a compound described herein or a pharmaceutically acceptable composition comprising said compound.

Another aspect relates to inhibiting DNMT activity in a biological sample, which method comprises contacting the biological sample with a compound described herein, or a pharmaceutically acceptable composition comprising said compound.

Another aspect relates to a method of inhibiting DNMT activity in a patient, which method comprises administering to the patient a compound described herein or a pharmaceutically acceptable composition comprising said compound.

Pharmaceutical compositions suitable for use in the present invention include compositions wherein the active ingredients are contained in an amount sufficient to achieve the intended purpose, e.g., the modulation of DNMT activity and/or the treatment or prevention of a DNMT-related disorder. More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein. For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the DNMT activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUTICS, Ch. 3, 9^{th} ed., Ed. by Hardman, J. et al., McGraw-Hill, New York City, 1996, p. 46.)

Dosage amount and interval may be adjusted individually to provide plasma levels of the active species which are sufficient to maintain the kinase modulating effects. These plasma levels are referred to as minimal effective concentrations (MECs). The MEC varies for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations. Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of the present invention may range from approximately 2.5 mg/m² to 1500 mg/m² per day. Additional illustrative amounts range from 0.2-1000 mg/qid, 2-500 mg/qid, and 20-250 mg/qid.

In cases of local administration or selective uptake, the effective local concentration of the drug may not be related to plasma concentration, and other procedures known in the art may be employed to determine the correct dosage amount and interval. The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug

Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

As mentioned above, the compounds and compositions of the invention will find utility in a broad range of diseases and conditions mediated by DNMTs, including diseases and conditions mediated by DNMT activity. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

The compounds of the invention can be used in combination with one or more other chemotherapeutic agents. The dosage of the inventive compounds may be adjusted for any drug-drug reaction. In one embodiment, the chemotherapeutic agent is selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that the above method can be carried out in combination with include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of antimetabolite chemotherapeutic agents that the above method can be carried out in combination with include, without limitation, folic acid analogs, e.g., methotrexate (used in the treatment of acute lymphocytic leukemia, choriocarcinoma, mycosis fungiodes, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents that the above method can be carried out in combination with include, without limitation, the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of useful COX-II inhibitors include VIOXX, CELEBREX (celecoxib), valdecoxib, paracoxib, rofecoxib, and Cox 189.

Examples of useful matrix metalloproteinase inhibitors are described in International Patent Publications WO 96/33172, WO 96/27583, WO 98/07697, WO 98/03516, WO 98/34918, WO 98/34915, WO 98/33768, WO 98/30566, WO 90/05719, WO 99/52910, WO 99/52889, and WO 99/29667, European Patent Application Nos. 97304971.1, 99302232.1, and 99308617.2, European Patent Publications 606,046, 780,386, and 931,788, PCT International Application No. PCT/IB98/01113, Great Britain patent application number 9912961.1, and U.S. Pat. Nos. 5,863,949, and 5,861,510, all of which are incorporated herein in their entireties by reference. Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Other anti-angiogenesis agents, other COX-II inhibitors and other MMP inhibitors, can also be used in the present invention.

The compounds of the invention can also be used with other signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970, WO 98/14451, WO 98/02434, and U.S. Pat. No. 5,747,498, and such substances can be used in the present invention as described herein. EGFR-inhibiting agents include, but are not limited to, the monoclonal antibodies C225 and anti-EGFR 22Mab (ImClone Systems, Inc., New York, N.Y.), the compounds erlotinib (OSI Pharmaceuticals, Inc., Melville, N.Y.), ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.). These and other EGFR-inhibiting agents can be used in the present invention.

VEGF inhibitors, for example SU-5416 and SU-6668 (Sugen Inc., South San Francisco, Calif.), can also be combined with an inventive compound. VEGF inhibitors are described in, for example, International Publications WO 01/60814, WO 99/24440, WO 95/21613, WO 99/61422, WO 99/10349, WO 97/32856, WO 97/22596, WO 98/54093, WO 98/02438, WO 99/16755, WO 01/60814, WO 98/50356, and WO 98/02437, PCT International Application PCT/IB99/00797, and U.S. Pat. Nos. 5,834,504, 5,883,113, 5,886,020, and 5,792,783, all of which are incorporated herein in their entireties by reference. Other examples of some specific VEGF inhibitors useful in the present invention are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present invention as described herein. Further, pErbB2 receptor inhibitors, such as GW-282974 (Glaxo Wellcome plc), and the monoclonal antibodies AR-209 (Aronex Pharmaceuticals Inc., The Woodlands, Tex.) and 2B-1 (Chiron), can furthermore be combined with an inventive compound, for example, those indicated in International Patent Publications WO 98/02434, WO 99/35146, WO 99/35132, WO 98/02437, WO 97/13760, and WO 95/19970, and U.S. Pat. Nos. 5,587,458, and 5,877,305, which are all hereby incorporated herein in their entireties by reference. ErbB2 receptor inhibitors useful in the present invention are also described in U.S. Pat. No. 6,284,764, incorporated in its entirety herein by reference. The erbB2 receptor inhibitor compounds and substance described in the aforementioned PCT applications, U.S. patents, and U.S. provisional applications, as well as other compounds and substances that inhibit the erbB2 receptor, can be used with an inventive compound, in accordance with the present invention.

The compounds of the invention can also be used with other agents useful in treating cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, of U.S. Pat. No. 6,258,824.

The above method can also be carried out in combination with radiation therapy, wherein the amount of an inventive compound in combination with the radiation therapy is effective in treating the above diseases. Techniques for administering radiation therapy are known in the art, and these techniques can be used in the combination therapy described herein. The administration of the compound of the invention in this combination therapy can be determined as described herein.

### EXAMPLES

The invention will be further understood upon consideration of the following non-limiting Examples. Abbreviations used below are ACN=acetonitrile; DCM=dichloromethane or methylenechloride; MeOH=methanol; EtOH=ethanol; DMSO=dimethylsulfoxide; DIPEA=N,N-diisopropylethylamine; DMAP=4-(dimethylamino)pyridine; HATU=2-(1H-7-azabenzotriazole-1-yl)-1,1,3,3-tetramethyluronium hexafluorophosphate; Boc=tertiary-butyloxycarbamate; HBTU=2-(1H-benzotriazole-1-yl)-1,1,3,3-tetramethyluronium hexafluorophosphate; TEA=triethylamine; THF=tetrahydrofuran; mcpba=meta-chloroperoxybenzoic acid; min, h, d=minutes, hours, days; rt or RT=room temperature; SM=starting material; NMR=nuclear magnetic resonance; TLC=thin layer chromatography.

### Chemistry

Compounds of the invention may be made by one of ordinary skill in the chemical arts using conventional synthetic procedures, as well as by the general reaction schemes and examples described below. Without specific statement, all solvents and reagents were available from SIGMA-Aldrich or VWR Chemicals and used as supplied or purified by standard laboratory methods as required. NMR spectra were recorded on a Varian Unity plus 300 at 300 MHz (¹H) at 25° C. Chemical shifts are reported in ppm and referenced internally to residual CHCl₃ for (d 7.26) or CH₃OH for (d 3.33). Low resolution mass spectrometry was performed by The Mass Spectrometry and Proteomics Core facility at the University of Utah. Flash chromatography was performed on Combifalsh (Yamazen) with normal phase silica gel column (RediSep) and CH₂Cl₂/CH₃OH solvent system. TLC used pre-coated silica gel aluminum sheets.

### General Synthesis

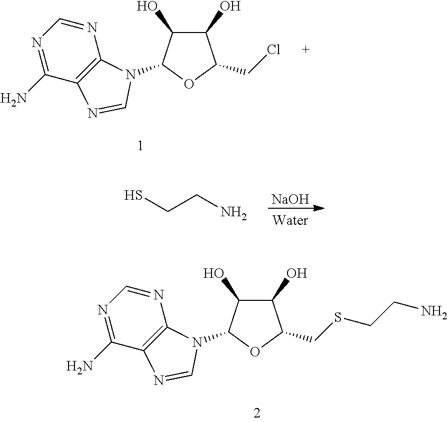

Cysteamine (89 mg, 1.155 mmol) was dissolved in a mixture of water (1.3 mL) and 1N NaOH (700 mL, 0.700 mmol). Nitrogen was then bubbled through the solution for 30 min. Intermediate 1 (100 mg, 0.350 mmol) was then added and the mixture was heated to reflux for 2 h. The reaction was cooled to rt and the water stripped off in vacuo. The crude product was then purified on prep TLC (20% MeOH in DCM with 2% NH₄OH) to give Intermediate 2 as a white solid. ¹H NMR (400 MHz DMSO-d₆) 8.35 (s, 1H), 8.14 (s, 1H), 7.27 (s, 2H), 5.88 (d, 1H), 4.74 (t, 1H), 4.13 (t, 1H), 3.89 (q, 1H), 2.86 (m, 4H), 2.78 (m, 2H).

### Example 1

### (2S,3S,4R,5R)-2-(6-Amino-9H-purin-9-yl)-5-(((2-((4-chloropyrimidin-2-yl)amino)ethyl)thio)methyl)tetrahydrofuran-3,4-diol (E1) and

### Example 2

### (2S,3S,4R,5R)-2-(6-Amino-9H-purin-9-yl)-5-(((2-((2-chloropyrimidin-4-yl)amino)ethyl)thio)methyl)tetrahydrofuran-3,4-diol (E2)

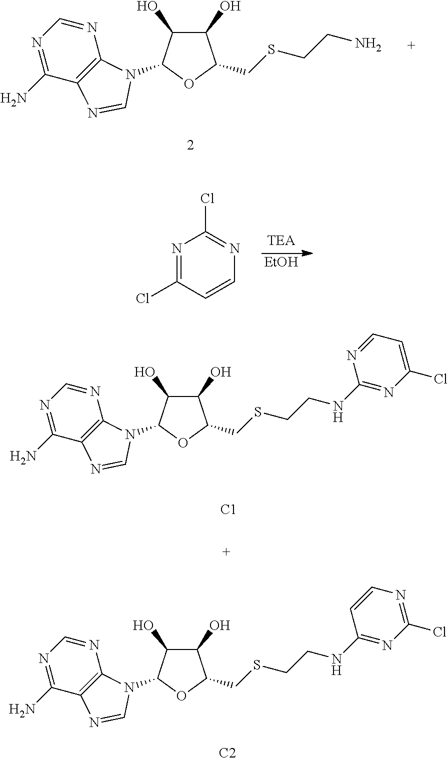

Triethylamine (152 μL, 1.1 mmol), Intermediate 2 (250 mg, 0.73 mmol) and 2,4-dichloropyrimidine (108 mg, 0.73 mmol) were added to 5 mL of ethanol. The reaction mixture was heated to reflux for 2.5 h, after which it was cooled to rt and the solvent removed in vacuo to give a crude oil that was purified via normal phase chromatography (0-10% MeOH in DCM). The two products both eluted at 10% MeOH with the minor product (EXAMPLE 1) being the first to elute. The major product (EXAMPLE 2) was 95% pure from the column with the only impurity being triethyl ammonium chloride, however, EXAMPLE 1 contained a large amount of triethylammonium chloride. To further purify EXAMPLE 1, it was triturated in MeOH then filtered off giving a pure white solid that was shown to be >95% by NMR

Example 1: 1H NMR (400 MHz, MeOH-d [symbol] 4) 8.51 (s, 1H), 8.39 (s, 1H), 8.13 (d, 1H), 6.62 (d, 1H), 6.07 (d, 1H), 4.74 (t, 1H), 4.34 (t, 1H), 4.25 (q, 1H), 3.54 (m, 2H), 3.02 (m, 2H), 2.79 (m, 2H)

Example 2: 1H NMR (400 MHz, MeOH-d [symbol] 4) 8.31 (s, 1H), 8.22 (s, 1H), 7.83 (d, 1H), 6.37 (d, 1H), 6.01 (d, 1H), 4.80 (t, 1H), 4.35 (t, 1H), 4.23 (q, 1H), 3.56 (m, 2H), 3.03 (m, 2H), 2.79 (m, 2H)

### Example 3

### (2S,3S,4R,5R)-2-(6-Amino-9H-purin-9-yl)-5-(((2-(pyrimidin-4-ylamino)ethyl)thio)methyl)tetrahydrofuran-3,4-diol (E3)

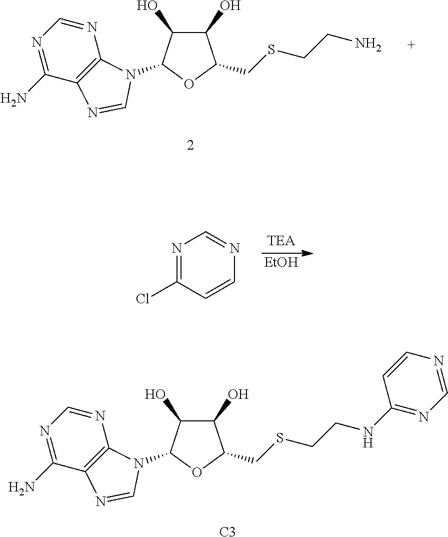 [SMILES: "EtOH" ; NCCSC[C@@H]1O[C@H](n2cnc3c(N)ncnc32)[C@@H](O)[C@H]1O ; Clc1ccncn1 ; Nc1ncnc2c1ncn2[C@H]1O[C@@H](CSCCNc2ccncn2)[C@H](O)[C@@H]1O ; "C3"]

Triethylamine (51 μL, 0.37 mmol), Intermediate 2 (80 mg, 0.25 mmol) and 4-chloropyrimidine HCl (37 mg, 0.25 mmol) were added to 3 mL of ethanol. The reaction mixture was heated to reflux for 24 h, after which it was cooled to rt and the solvent removed in vacuo. The crude product was then purified via normal phase chromatography (0-15% MeOH in DCM) to give EXAMPLE 3 as a white solid.

1H NMR (400 MHz, DMSO-d6) 8.38 (s, 1H), 8.36 (s, 1H), 8.15 (s, 1H), 7.99 (d, 1H), 7.54 (bs, 1H), 7.30 (s, 1H), 6.44 (d, 1H), 5.89 (d, 1H), 5.52 (d, 1H), 5.34 (d, 1H), 4.74 (q, 1H), 4.15 (q, 1H), 4.03 (q, 1H), 3.43 (m, 2H), 2.92 (m, 2H), 2.68 (m, 2H)

### Example 4

### (2S,3S,4R,5R)-2-(6-Amino-9H-purin-9-yl)-5-(((2-(6-chloropyrimidin-4-yl)amino)ethyl)thio)methyl)tetrahydrofuran-3,4-diol (E4)

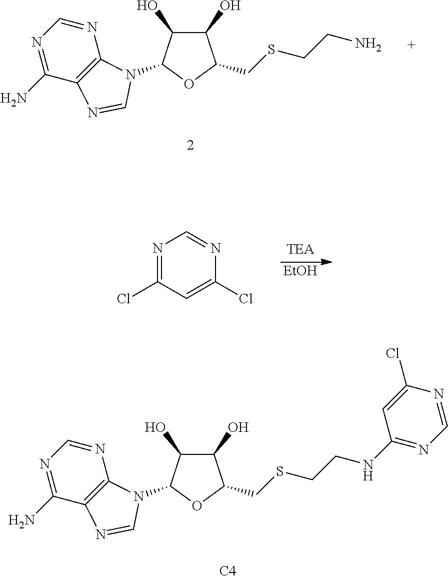 [SMILES: "EtOH" ; NCCSC[C@@H]1O[C@H](n2cnc3c(N)ncnc32)[C@@H](O)[C@H]1O ; Clc1cc(Cl)ncn1 ; Nc1ncnc2c1ncn2[C@H]1O[C@@H](CSCCNc2cc(Cl)ncn2)[C@H](O)[C@@H]1O ; "C4"]

Triethylamine (51 μL, 0.37 mmol), Intermediate 2 (80 mg, 0.25 mmol) and 4,6-dichloropyrimidine (37 mg, 0.25 mmol) were added to 3 mL of ethanol. The reaction mixture was heated to reflux for 2.5 h, after which it was cooled to rt and the solvent removed in vacuo. The crude product was then purified via normal phase chromatography (0-15% MeOH in DCM) to give EXAMPLE 4 as a white solid.

1H NMR (400 MHz, DMSO-d6) 8.34 (s, 1H), 8.25 (bs, 1H), 8.15 (s, 1H), 7.82 (bs, 1H), 7.28 (s, 2H), 6.52 (bs, 1H), 5.89 (d, 1H), 5.49 (d, 1H), 5.31 (d, 1H), 4.74 (m, 1H), 4.15 (m, 1H), 4.03 (m, 1H), 3.47 (m, 2H), 2.92 (m, 2H), 2.69 (m, 2H)

### Example 5

### N-(2-((((2R,3R,4S,5S)-5-(6-Amino-9H-purin-9-yl)-3,4-di hydroxytetrahydrofuran-2-yl)methyl)thio)ethyl)acrylamide (E5)

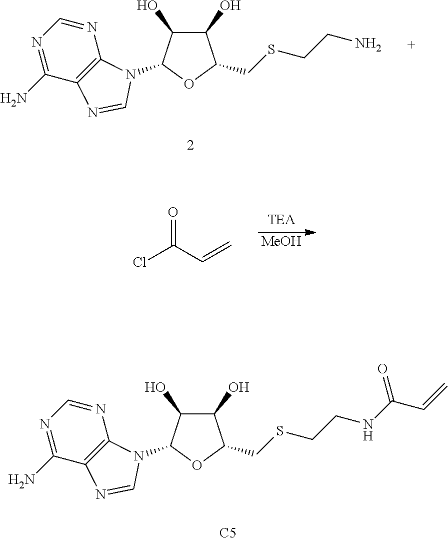

Intermediate 2 (87 mg, 0.27 mmol) was dissolved in 1.5 mL of MeOH. Triethylamine (82 μL, 0.59 mmol) was then added followed by acryloyl chloride (24 μL, 0.29 mmol). The reaction was stirred at rt for 30 min. The solvent was then removed in vacuo and the crude product purified via normal phase MPLC (0-15% MeOH in DCM) to give EXAMPLE 5 as a white solid.

1H NMR (400 MHz, DMSO-d6) 8.35 (s, 1H), 8.21 (bt, 1H), 8.16 (s, 1H), 7.29 (s, 2H), 6.23-6.04 (m, 2H), 5.89 (d, 1H), 5.57 (d, 1H), 5.49 (d, 1H), 5.31 (m, 1H), 4.74 (m, 1H), 4.14 (m, 1H), 4.02 (m, 1H), 3.28 (m, 2H), 2.89 (m, 2H), 2.61 (m, 2H)

### Protocols for DNMT Inhibition Assays

Catalytic domains of human DNMT 3a/3L enzyme were produced by Accelagen. Human full-length DNMT1 enzyme purchased from BPS Biosciences. Poly(dldC) DNA (Sigma) was used at 0.002 mg/mL. H3 SAM (Perkin Elmer) was used at 500 nM. DNA, DNMT enzyme, and inhibitors were mixed with assay buffer (20 mM Tris pH8, 5% glycerol, 1 mM EDTA, 100 μg/mL BSA, 1 mM DTT, 50 mM NaCl) and pre-incubated without SAM for 30 min. H3 SAM was then added to initiate the methylation reaction which continues at 37° C. for 1 h. Reaction mixture was then transferred to a filter plate (Millipore Multiscreen HTS DE), then washed with 50 mM H2PO₄ and 95% Ethanol. Scintillation counts were measured on the Perkin Elmer Microbeta scintillation counter. DNMT inhibition activity for EXAMPLES 1 to 5 is shown in Table 1 below:

TABLE 1

| Example | DNMT1 IC50 (μM) | DNMT 3a/3I IC50 (μM) |
|---|---|---|
| 1 | >300 | 3 |
| 2 | Not tested | 95 |
| 3 | Not tested | 26 |
| 4 | 48 | 60 |
| 5 | 49 | 24 |

1. A compound of formula (I): 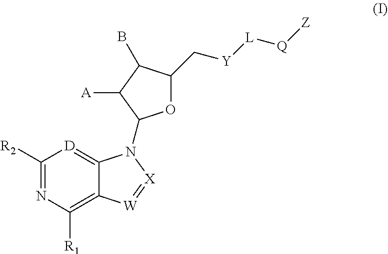
or pharmaceutically acceptable salts thereof; wherein:
A and B independently represent hydrogen, halogen or OH;
R₁ represents hydrogen or NR₃R₄;
R₃ and R₄ independently represent hydrogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups;
R₂ represents hydrogen, halogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups;
D represents CH or N;
W represents CH, N, C-alkyl or C-halogen;
X represents CH, N, C-alkyl or C-halogen;
Y and Q independently represent a bond, CR₅R₆, O, S or NR₇;
R₅, R₆ and R₇ independently represent hydrogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups;
L represents a bond, —(CH₂)ₙ—, 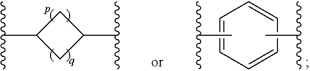
n represents an integer selected from 1 to 5;
p and q independently represents an integer selected from 0 to 3;
Z represents 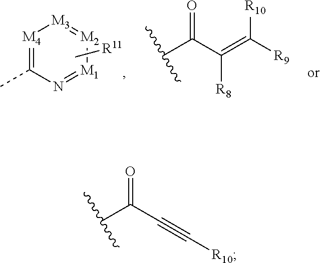
M₁, M₂, M₃ and M₄ independently represent CH or N;
R₈, R₉ and R₁₀ independently represent hydrogen, alkyl, cycloalkyl, heterocyclyl, aryl, heteroaryl, wherein cycloalkyl, heterocyclyl, aryl, heteroaryl, at each occurrence, may be optionally substituted by one or more Rₐ groups; and
R₁₁ represents hydrogen, fluorine or chlorine;
Rₐ represents halogen, C₁₋₆ alkyl, C₂₋₆ alkenyl, C₂₋₆ alkynyl, —(CH₂)₀₋₄—C₃₋₈ cycloalkyl, —(CH₂)₀₋₄—C₃₋₈ cycloalkenyl, —(CH₂)₀₋₄-phenyl, —(CH₂)₀₋₄-(heterocyclyl), —(CH₂)₀₋₄-(heteroaryl), —(CR^{x}R^{y})₀₋₄—O—R^{z}, —O—(CR^{x}R^{y})₁₋₄—OR^{z}, haloC₁₋₆ alkyl, haloC₁₋₆ alkoxy, C₁₋₆ alkanol, ═O, ═S, nitro, Si(R^{x})₄, —(CH₂)₀₋₄—CN, —S(O)₀₋₂—R^{x}, —C(═O)R^{x}, —(CR^{x}R^{y})₀₋₄—C(═O)OR^{z}, —(CR^{x}R^{y})₀₋₄—O—C(═O)—R^{z}, —(CR^{x}R^{y})₀₋₄—C(═O)NR^{x}R^{y}, —(CH₂)₀₋₄—NR^{x}C(═O)R^{y}, —(CH₂)₀₋₄—OC(═O)NR^{x}R^{y}, —(CH₂)₀₋₄—NR^{x}C(═O)OR^{y}, —(CH₂)₀₋₄—NR^{x}R^{y}, —NR^{x}—(CH₂)₀₋₄—R^{z}, —(CH₂)₀₋₄—O—C(═O)—C₁₋₄alkyl-NR^{x}R^{y}, —(CH₂)₀₋₄—NR^{x}—(CH₂)₁₋₄—O—C(═O)—R^{z}, —(CH₂)₀₋₄—NR^{x}—(CH₂)₀₋₄—SO₂—R^{y}, —(CH₂)₀₋₄—NH—SO₂—NR^{x}R^{y}, —(CH₂)₀₋₄—SO₂NR^{x}R^{y} and —P(═O)(R^{x})₂ groups;
R^{x}, R^{y} and R^{z} independently represent hydrogen, C₁₋₆ alkyl, C₂₋₆ alkenyl, C₂₋₆ alkynyl, —(CH₂)₀₋₄—C₃₋₈ cycloalkyl, —(CH₂)₀₋₄—C₃₋₈ cycloalkenyl, —(CH₂)₀₋₄-(heterocyclyl), —(CH₂)₀₋₄-(heteroaryl), C₁₋₆ alkanol optionally substituted with one or more halo, —C(═O)OC₁₋₆ alkyl, hydroxy, C₁₋₆ alkoxy, haloC₁₋₆ alkyl, —(CH₂)₁₋₄—O—C₁₋₆alkyl, —C(═O)—(CH₂)₁₋₄—C₁₋₆ alkoxy, —C(═O)—C₁₋₆alkyl, —(CH₂)₀₋₄—CN, C₁₋₆ alkyl-N(H)₂₋ᵣ(C₁₋₆alkyl)ᵣ, —N(H)₂₋ᵣ(C₁₋₆alkyl)ᵣ, —C(═O)—N(H)₂₋ᵣ(C₁₋₆alkyl)ᵣ, —(CH₂)₀₋₄—NH—SO₂—N(H)₂₋ᵣ(C₁₋₆alkyl)ᵣ, —(CH₂)₀₋₄—N(C₁₋₄alkyl)-SO₂—N(H)₂₋ᵣ(C₁₋₆alkyl)ᵣ and —(CH₂)₀₋₄—O—C(═O)—C₁₋₄alkyl-N(H)₂₋ᵣ(C₁₋₆alkyl)ᵣ, and when attached to nitrogen or carbon or phosphorus or silicon atom R^{x} and R^{y} may join to form a 3-7 membered ring optionally containing a one or two heteroatoms selected from O, N, S and oxidised forms of N or S; and
r represents an integer selected from 0 to 2.

2. A compound as defined in claim 1, wherein A and B both represent OH.

3. A compound as defined in claim 1, wherein R₁ represents NH₂.

4. A compound as defined in claim 1, wherein R₂ represents hydrogen.

5. A compound as defined in claim 1, wherein D represents N.

6. A compound as defined in claim 1, wherein W represents N.

7. A compound as defined in claim 1, wherein X represents CH.

8. A compound as defined in claim 1, wherein Y represents S.

9. A compound as defined in claim 1, wherein Q represents NH.

10. A compound as defined in claim 1, wherein R₇ represents hydrogen.

11. A compound as defined in claim 1, wherein L represents —(CH₂)₂—.

12. A compound as defined in claim 1, wherein Z represents

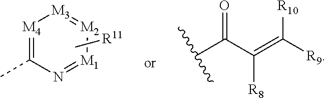

13. A compound as defined in claim 12, wherein Z represents

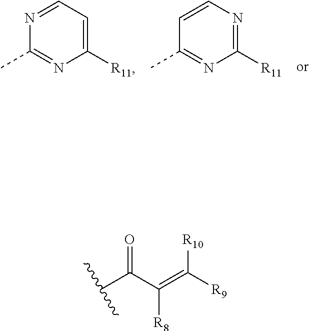

14. A compound as defined in claim 12, wherein R₁₁ represents hydrogen or chlorine.

15. A compound as defined in claim 12, wherein R₁₁ represents chlorine.

16. A compound as defined in claim 12, wherein R₈, R₉ and R₁₀ each represent hydrogen.

17. A compound of formula (IA) or a pharmaceutically acceptable salt thereof: 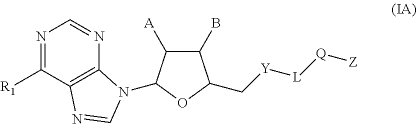
wherein R₁, A, B, Y, L, Q and Z are as defined in claim 1.

18. A compound of formula (IB) or a pharmaceutically acceptable salt thereof: 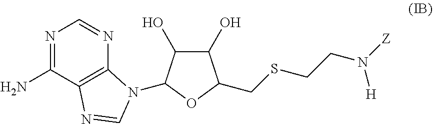
wherein Z is as defined in claim 1.

19. A compound of formula (I) as defined in claim 1, which is selected from:
(2S,3S,4R,5R)-2-(6-amino-9H-purin-9-yl)-5-(((2-((4-chloropyrimidin-2-yl)amino)ethyl)thio)methyl)tetrahydrofuran-3,4-diol;
(2S,3S,4R,5R)-2-(6-amino-9H-purin-9-yl)-5-(((2-((2-chloropyrimidin-4-yl)amino)ethyl)thio)methyl)tetrahydrofuran-3,4-diol;
(2S,3S,4R,5R)-2-(6-amino-9H-purin-9-yl)-5-(((2-(pyrimidin-4-ylamino)ethyl)thio)methyl)tetrahydrofuran-3,4-diol;
(2S,3S,4R,5R)-2-(6-amino-9H-purin-9-yl)-5-(((2-((6-chloropyrimidin-4-yl)amino)ethyl)thio)methyl)tetrahydrofuran-3,4-diol;
N-(2-((((2R,3R,4S,5S)-5-(6-amino-9H-purin-9-yl)-3,4-dihydroxytetrahydrofuran-2-yl)methyl)thio)ethyl)acrylamide;
or pharmaceutically acceptable salts thereof.

20. A pharmaceutical composition comprising a compound of formula (I) as defined in claim 1.

21. A compound as defined in claim 1 for use in therapy.

22. A compound as defined in claim 1, for use in treating cancer or hyperproliferative disorders.

23. A method of treating cancer or hyperproliferative disorders by administering an effective amount of the compound according to claim 1.

24. The compound or method of claim 22, wherein the cancer is of colon, breast, stomach, prostate, pancreas, or ovarian tissue.

25. A process for preparing a compound of formula (I) as defined in claim 1 which comprises:
(a) preparing a compound of formula (I) wherein Q represents NH by reacting a compound of formula (II): 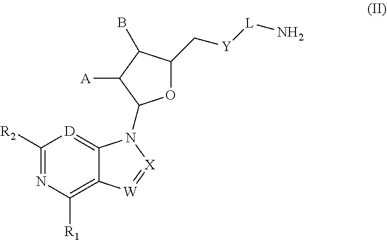
wherein R₁, R₂, D, X, W, A, B, Y and L are as defined in claim 1, with a compound of formula Z-L₁ wherein Z is as defined in claim 1 and L₁ represents a suitable leaving group such as chlorine;
(b) deprotection of a protected derivative of a compound of formula (I);
(c) interconversion of a compound of formula (I) or protected derivative thereof to a further compound of formula (I) or protected derivative thereof; and
(d) optional formation of a pharmaceutically acceptable salt of a compound of formula (I).
